(19) United States
(12) Patent Application Publication
(10) Pub. No.: US 2025/0009989 A1
(43) Pub. Date: Jan. 9, 2025

(54) Rotator for a medicament delivery device

(71) Applicant: SHL Medical AG, Zug (CH)

(72) Inventors: Oscar Alexandersson, Nacka Strand (SE); Johan Zander, Nacka Strand (SE); Daniel Carlsson, Nacka Strand (SE)

(21) Appl. No.: 18/706,954

(22) PCT Filed: Oct. 21, 2022
(86) PCT No.: PCT/EP2022/079361

§ 371 Date: May 2, 2024

(30) Foreign Application Priority Data
Nov. 3, 2021 (EP) 21206324.2

(51) Int. Cl. A61M 5/34; A61M 5/20

(52) U.S. Cl. CPC A61M 5/347; A61M 2005/2006

(57) ABSTRACT

The present disclosure provides a rotator for a medicament delivery device. The rotator includes a tubular body extending from a proximal end to a distal end in an axial direction relative to an axis and in a circumferential direction relative to the axis and one or more ridges extending from a surface of the tubular body. The one or more ridges define a track on the surface of the tubular body, the track extending in the axial direction from a distal end of the track to a proximal end of the track. The track comprises a first pathway and a second pathway connected to the first pathway. The first pathway is at the proximal end of the track and the second pathway is at the distal end of the track. The first pathway comprises two sections adjacent to one another in the circumferential direction. The first section is delimited in the distal direction by a portion of the one or more ridges, and the second section is connected to the second pathway at a distal end of the second section.

### CROSS-REFERENCE TO RELATED APPLICATIONS

The present application is a U.S. National Phase Application pursuant to 35 U.S.C. § 371 of International Application No. PCT/EP2022/079361 filed Oct. 21, 2022, which claims priority to European Patent Application No. 21206324.2 filed Nov. 3, 2021. The entire disclosure contents of these applications are herewith incorporated by reference into the present application.

### TECHNICAL FIELD

The invention concerns medicament delivery devices, and particularly rotators for medicament delivery devices.

### BACKGROUND

Medicament delivery devices such as those described in WO 2011/123024 have already been very commercially successful due to a combination of properties such as robustness, simplicity and usability. Nevertheless, the applicant has appreciated that there is still further scope for improvement of the rotator in medicament delivery devices such as those described in WO 2011/123024.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

An aspect concerns a rotator for a medicament delivery device, the rotator comprising: a tubular body extending from a proximal end to a distal end in an axial direction relative to an axis and in a circumferential direction relative to the axis; and one or more ridges extending from a surface of the tubular body, the one or more ridges defining a track on the surface of the tubular body, the track extending in the axial direction from a distal end of the track to a proximal end of the track, the track comprising a first pathway and a second pathway connected to the first pathway, wherein the first pathway is at the proximal end of the track and the second pathway is at the distal end of the track, wherein the first pathway comprises two sections adjacent to one another in the circumferential direction, and wherein the first section of the two sections is delimited in the distal direction by a portion of the one or more ridges, and the second section of the two sections is connected to the second pathway at a distal end of the second section. Delimiting the first section of the two sections in the distal direction by a portion of the one or more ridges can stop a protrusion of a medicament delivery member guard of a medicament delivery device that contains the rotator from moving in the distal direction from the first section of the first pathway.

Optionally, the one or more ridges comprise a protrusion extending in the proximal direction, the protrusion extending between the first section of the first pathway and the second section of the first pathway. This can help stop the protrusion of the medicament delivery member guard from accidentally leaving the first section of the first pathway.

Optionally, the portion of the one or more ridges extends in the circumferential direction.

Optionally, the portion of the one or more ridges extends perpendicular to the axis.

Optionally, a proximal face of the portion of the one or more ridges is curved. This can help guide the protrusion of the medicament delivery member guard when the protrusion is moving from the first portion of the first pathway to the second portion of the first pathway (for example when the cap is removed from a device containing the rotator).

Optionally, the portion of the one or more ridges (30) is a first portion. This portion can stop the protrusion of the medicament delivery member guard from moving in the distal direction from this portion. Optionally, the one or more ridges comprises a second portion arranged proximal to the first portion.

Optionally, the second portion is angled relative to the axis. Optionally, the second portion (73) extends from a proximal end to a distal end, and wherein the proximal end of the second portion is adjacent to the second section of the first pathway. Optionally, the second portion is linear.

Optionally, the first pathway comprises a third section, and the third section is adjacent to the second pathway, and the third section connects the second section to the second pathway. Optionally, the third section extends from a proximal end to a distal end and reduces in width towards the distal end of the third section.

Optionally, the rotator comprises a third pathway connected to the second pathway, wherein the third pathway is at the proximal end of the track. Optionally, the first pathway and the third pathway are separated by at least one of the one or more ridges.

Optionally, the one or more ridges extend from an outer surface of the rotator.

Another aspect concerns a medicament delivery device comprising a rotator as described above. Optionally, the medicament delivery device is an autoinjector. Optionally, the medicament delivery device comprises a medicament delivery member guard, the medicament delivery member guard comprising a protrusion, wherein the protrusion is in the track of the rotator. Optionally, the protrusion is configured to be in the first portion of the first pathway prior to device activation, in the second portion of the first pathway after device activation and in the second pathway during medicament delivery. Optionally, the protrusion is configured to be in the third pathway after medicament delivery.

Another aspect concerns a rotator for a medicament delivery device, the rotator comprising a track to guide a protrusion of a medicament delivery member guard, wherein the track comprises a section that is delimited in the distal direction to limit movement of the medicament delivery member guard in the distal direction prior to medicament delivery device activation.

Another aspect concerns a method of activating a medicament delivery device, comprising the step of removing a cap from a medicament delivery device so that a medicament delivery member guard of the medicament delivery device moves in a proximal direction relative to a housing of the medicament delivery device, thereby rotating a rotator of the medicament delivery device from a locked position to an activated position.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the accompanying drawings, in which:

FIG. 1 is a perspective view of an example rotator.

FIG. 2 is a side view of the rotator of FIG. 1.

FIG. 3 is a perspective view of the rotator of FIG. 1.

FIG. 4 is a perspective view of another example rotator.

FIG. 5 shows a further perspective view of the rotator of FIGS. 1 to 3.

FIG. 6 shows a simplified line diagram of another example of the track of a rotator.

FIGS. 7 to 10 shows side views of the rotator of FIG. 1 with a protrusion of a medicament delivery member guard in different pre-injection positions.

FIGS. 11 to 13 show a cross-section view of a front portion of a medicament delivery device before, during and after cap removal respectively.

FIG. 14 shows a perspective view of an example housing, cap and medicament delivery member guard that could be used with the rotators shown in FIGS. 1 to 10.

### DETAILED DESCRIPTION

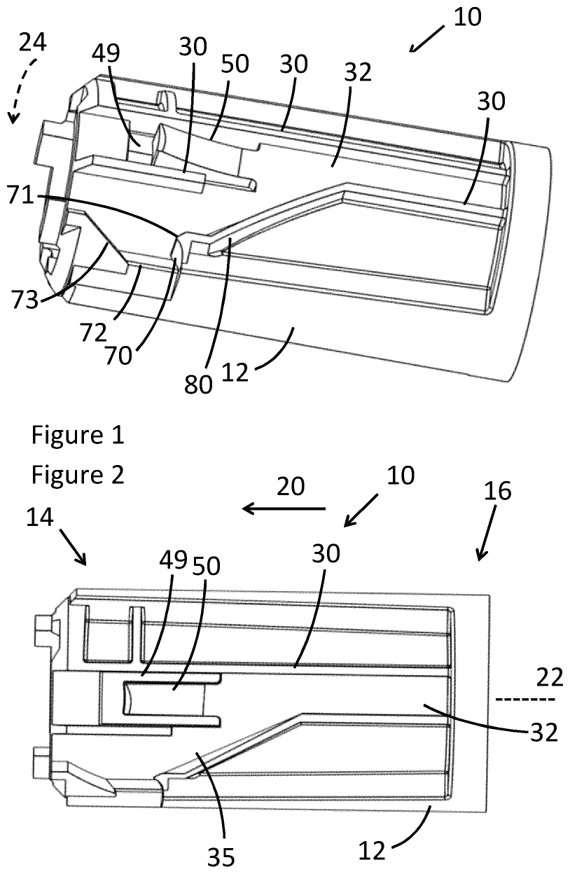

FIGS. 1 to 3 and 5 show views of an example of a rotator 10 for a medicament delivery device. The rotator 10 comprises a tubular body 12 extending from a proximal end 14 to a distal end 16 in an axial direction 20. Ridges 30 extend from a surface of the tubular body 12, with the ridges 30 defining a track (labyrinth) 32 on the surface of the tubular body 12, the track 32 extending in the axial direction 20 from a distal end of the track 32 to a proximal end of the track 32. The track 32 comprises one pathway 36 at the distal end of the track 32 and two pathways 34, 38 at the proximal end of the track 32. The typical shape of the pathways will be described in more detail with reference to another similar example shown in FIG. 6. The two pathways 34, 38 at the proximal end of the track 32 are separated by a part of the ridge 30.

An optional tongue 50 is also shown in an opening 49 in the surface of the track 32 in FIGS. 1 and 2. The structure and purpose of the tongue 50 is described in more detail below.

As described in more detail below with reference to FIG. 6, the track 32 comprises a first pathway comprising a first section, a second section and a third section. The first section is adjacent to the second section on one side in the circumferential direction (this could be either side, depending on which way the rotator is designed to rotate relative to other medicament delivery components). At the distal end of the first section, a ridge portion 70 delimits the first section.

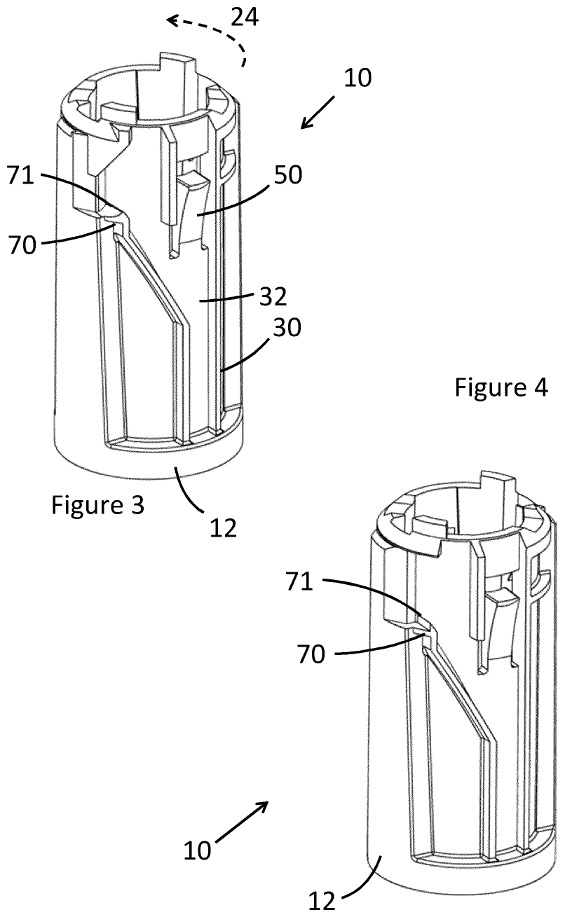

FIG. 4 shows an alternative rotator from a similar perspective to the perspective shown in FIG. 3 for the rotator described above. The rotator of FIG. 3 comprises a ridge portion 70 which is curved (more specifically, the proximal face of the ridge portion 70 curves), and the ridge also comprises a protrusion 71 extending into the track towards the proximal end of the rotator between the first section of the first pathway and the second section of the first pathway; this protrusion is adjacent to the ridge portion 70 and the ridge portion 80. This protrusion can make accidental activation harder. The alternative rotator of FIG. 4 comprises a ridge portion 70 which is linear. The ridge portion 70 could also extend in other directions, particularly when a protrusion 71 is provided to help avoid accidental activation. The ridge portion 70 extends in the circumferential direction, and may also be perpendicular to the axis 22 (when the ridge portion 70 is linear).

The first section of the first pathway is delimited by the second section of the first pathway on one side and by a ridge portion 72 on the other side, with the ridge portion 72 extending between the protrusion 71 and a ridge portion 73 in the axial direction 20. The ridge portion 73, which is optional, is linear and extends at an angle relative to both the axial direction and the circumferential direction, with the proximal end of the ridge portion 73 closer to (in this case adjacent to) the second section of the first pathway than the distal end of the ridge portion 73. For example, the angle of the ridge portion 73 relative to the axis is between 10 and 80 degrees, and preferably between 30 and 60 degrees. The ridge portion 73 can help guide the medicament delivery member guard (needle guard) from the first section of the first pathway to the second section of the first pathway. The ridge portion 72 is also optional, with a gap left in the ridge, or ridge portion 70 and ridge portion 73 could be directly connected together, for example.

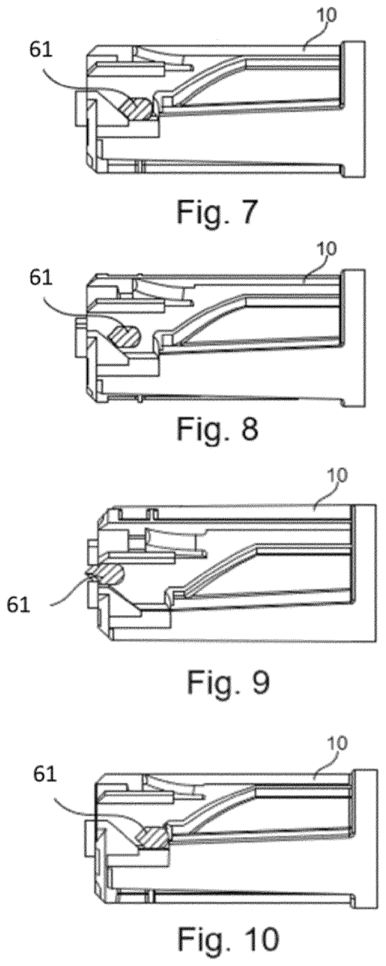

In FIGS. 7 to 10, a further view of the rotator 10 of FIGS. 1 and 2 can be seen. Part of a medicament delivery member guard 60 is also shown, namely a protrusion 61 of the medicament delivery member guard 60 shown in the track 32. FIG. 7 shows the relative positions after device assembly is completed. FIG. 8 shows the relative positions during activation of the device (e.g. activation by taking off the cap). FIG. 9 shows the relative positions after activation is completed but before use of the device for medicament delivery (injection) has been started. FIG. 10 shows the relative positions before activation if the device is dropped (for example dropped with the distal end of the device impacting the floor, thereby pushing the medicament delivery member guard in the distal direction relative to the rotator).

In FIG. 7, the position of the protrusion 61 in the rotator 10 in an assembled medicament delivery device can be seen—i.e. before use of the device. The protrusion is in the first portion of the first pathway (this is equivalent to initial position of the protrusion as shown in FIG. 6). In this particular example, the protrusion 61 is spaced apart from the ridge portion 70 in the axial direction and is in contact with the ridge portion 73 (this is optional and the protrusion 61 could be in contact with none, one of or both of the ridge portion 70 and the ridge portion 73). During activation of the device (in this case by removing the cap), the medicament delivery member guard moves in the proximal direction relative to the rotator. FIG. 8 shows an intermediate position during activation, and FIG. 9 shows the position after activation (this is equivalent to the intermediate position 62 of the protrusion as shown in FIG. 6). FIG. 10 shows the medicament delivery member guard impacting the ridge portion 70 after being pushed in the distal direction by the impact of the device being dropped, with the ridge portion 70 thereby stopping premature activation of the device.

It is optional that there is a gap between the ridge portion 70 and the protrusion 61 in the completed medicament delivery device. However, this gap between can be beneficial as it can allow for less strict tolerance margins, for example.

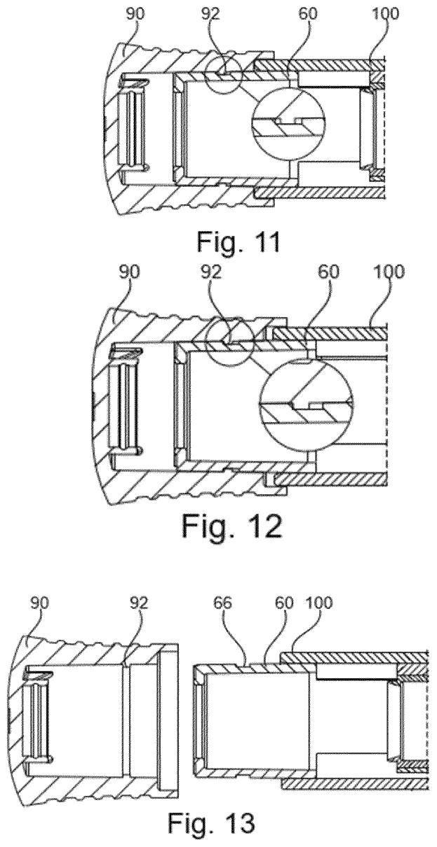

FIGS. 11 to 13 show an example of one way that the medicament delivery member guard could be moved in the proximal direction relative to the rotator to move the protrusion 61 from the first section of the first pathway to the second section of the first pathway. An optional cap 90, a housing 100 and a medicament delivery member guard 60 are shown-several other possible components, such as a medicament delivery member shield remover (needle shield remover) of the cap, are not shown for simplicity. The housing would typically be fixed in the axial direction relative to the rotator, so that movement of the medicament delivery member guard relative to the housing in the axial direction is equivalent to movement of the medicament delivery member guard relative to the rotator. In FIG. 11, the position of the cap 90, the housing 100 and the medicament delivery member guard 60 relative to one another in an assembled medicament delivery device is shown, before use of the device. A rib 92 of the cap extends towards the axis from an inner surface of the cap. The rib 92 extends into a corresponding groove 66 on the outer surface of the medicament delivery member guard. As a result, when the cap is pulled in the proximal direction relative to the housing 100, the cap pulls the medicament delivery member guard in the proximal direction into the position shown in FIG. 12, thereby moving the protrusion 61 of the medicament delivery member guard from the position shown in FIG. 7 to the position shown in FIG. 9. Once the medicament delivery member guard reaches the allowed limit within the device (for example due to being blocked from further proximal movement relative to the housing/rotator by a feature of the housing and/or a feature of the rotator), continued pulling of the cap in the proximal direction overcomes the resistance provided by the interaction between the rib 92 and the groove 66, thereby pulling the cap off the device (as shown in FIG. 13).

Various different approaches could be used to move the medicament delivery member guard in the proximal direction relative to the rotator; for example, instead of a rib 92 and a groove 66, a protrusion and a recess could instead be provided. The rib/protrusion could be on the medicament delivery member guard instead of on the cap, and the groove/recess could be on the cap instead of on the medicament delivery member guard. Multiple ribs/protrusions and/or grooves/recesses could be provided. Alternatively, other approaches such as a friction fit could be used.

The rotator described above is typically a standalone component in a medicament delivery device such as an autoinjector. An example of a type of medicament delivery device the rotator could be used in is described in WO 2011/123024, which is herein incorporated by reference, particularly with reference to FIGS. 1 and 2 and the corresponding description. Briefly, a medicament delivery device incorporating the rotator would comprise a housing (such as an outer housing), a medicament delivery member guard and the rotator. The rotator is able to move rotationally (in the circumferential direction) within the housing during use of the medicament delivery device. The medicament delivery member guard is able to move in the axial direction within the housing during use of the medicament delivery device. Typically, axial movement of the rotator relative to the housing would be restricted. Typically, rotational movement of the medicament delivery member guard relative to the housing would be restricted. The medicament delivery device also typically comprises a powerpack inside the housing, the powerpack comprising the rotator and a plunger rod, a medicament barrel inside the outer housing and a cap. The medicament barrel typically comprises a medicament container and a medicament delivery member such as a needle. Alternatively, a different medicament delivery member, such as a jet injector, could be used.

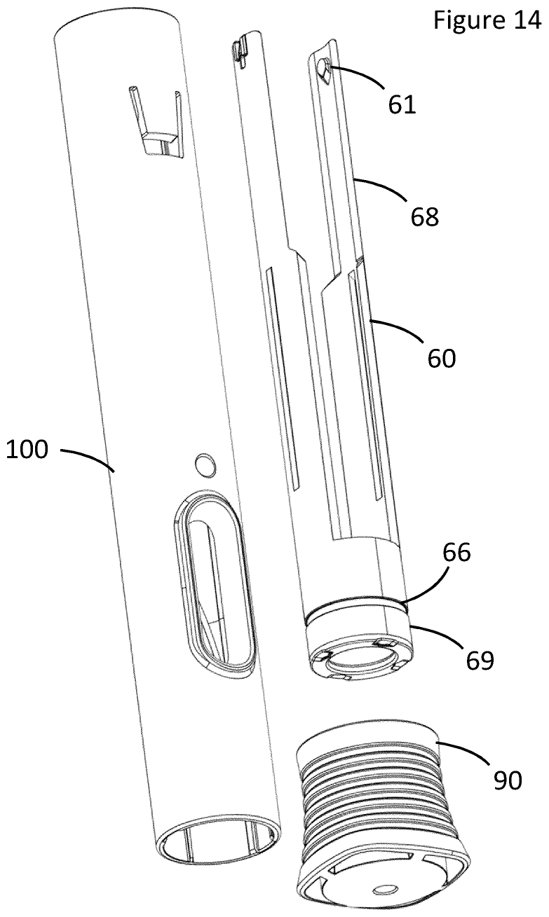

FIG. 14 shows an example of a medicament delivery member guard 60, a cap 90 and a housing 100 that could be used in the examples given above. The housing is tubular (in this case cylindrical) but could be another shape. The protrusion 61 and the groove 66 of the medicament delivery member guard 60 can be seen. The medicament delivery member guard comprises two distally extending arms 68 extending in the distal direction from a tubular body 69 (which in this case is cylindrical), with protrusions 61 on the distal end of each arm (only one of the protrusions is visible), although the medicament delivery member guard 60 could be a different shape or structure, with for example one, three or more arms instead of two arms. Various other optional features, including a ribbed outer surface on the cap and a window in the housing, can also be seen.

In a medicament delivery device comprising the rotator 10 and the medicament delivery member guard 60, the rotator 10 and medicament delivery member guard 60 can move relative to one another. The typical movement of the rotator relative to the medicament delivery member guard will now be described with reference to FIG. 6. FIG. 6 shows a simplified view of another example track 32. As mentioned above, the track 32 can be thought of as a combination of three separate pathways, namely a first pathway 34, a second pathway 36 and a third pathway 38, which are shown separated by dotted lines in FIG. 3. A transition portion 35 is considered to be part of the first pathway 34. A locking portion 33 is also considered to be part of the first pathway 34. The first pathway therefore comprises three portions, namely a first section (locking portion 33) (as described in more detail above), a second section or main portion, and a third section (transition portion 35).

The first and third pathways 34, 38 typically correspond to the two pathways at the proximal end of the track as described above, and the second pathway typically corresponds to the pathway at the distal end of the track as described above. The protrusion 61 will be described as ‘following’ the track/pathways, which in practice means that the protrusion is restricted to movement along the track/pathways by the ridges 30, and is either touching or near to the surface of the tubular body in the track as a result.

The transition portion 35 is a portion in which the width of the first pathway narrows in the circumferential direction, with the transition portion tapering (reducing) in width towards the distal end of the rotator. This transition portion is designed to guide the protrusion 61, and it is normally this portion that results in the rotation of the rotator as the medicament delivery member guard moves in the distal direction relative to the rotator, which is described in more detail below.

The protrusion 61 of the medicament delivery member guard 60 would initially be at the position shown in FIG. 6 in an assembled and unused medicament delivery device. The protrusion would then move to position 62 during cap removal. When an injection is subsequently carried out using the medicament delivery device, the medicament delivery member guard moves in the distal direction relative to other parts of the device (relative to the rotator and relative to an outer housing, for example). In other words, relative to the medicament recipient, the medicament delivery member guard remains stationary with the proximal end of the medicament delivery member guard against the dose delivery site while the other parts of the device move in the proximal direction towards the dose delivery site. As the medicament delivery member guard moves relative to the rotator, the protrusion 61 also moves relative to the rotator and is arranged in the track so that it follows the track, firstly along the first pathway (specifically the second portion and the third portion) and then along the second pathway. The medicament delivery member guard is typically rotationally fixed relative to other parts of the medicament delivery device (e.g. an outer housing, which will be used here as an example).

Roughly speaking, the protrusion is in the first pathway before injection, in the second pathway during injection and in the third pathway after injection, although the location of the protrusion during the precise transition points (from before to during to after injection) can vary somewhat depending on device design and use, so these transitions do not necessarily happen precisely when the protrusion travels from the first pathway to the second pathway and from the second pathway to third pathway—and indeed, injection completion is normally with the protrusion still in the second pathway. In more detail: immediately before injection, the protrusion is in the second section of the first pathway, and during the subsequent movement of the protrusion in the first pathway, the medicament delivery member guard moves axially relative to the outer housing and the rotator remains stationary relative to the outer housing. As the protrusion enters the transition portion 35 and transfers to the second pathway 36, the medicament delivery member guard continues to move axially relative to the outer housing, and the rotator also rotates (in the circumferential direction) relative to the outer housing and the medicament delivery member guard. The point at which the injection would start would typically be with the protrusion in the transition portion 35 and/or in the second pathway 36. The protrusion can optionally be used to provide an indication that the injection is starting—for example either that the injection is about to start, that it is starting, or it has just started, depending on the exact relative positions of the various parts of the medicament delivery device. This could be provided by a second tongue similar to the tongue 50 interacting with the protrusion, for example.

During the actual injection, the medicament delivery member guard, the rotator and the outer housing would typically be stationary relative to one another. Once the injection is finished (which is typically indicated by an end click generated elsewhere within the medicament delivery device), the medicament delivery device is removed from the injection site, and as a result the medicament delivery member guard will move axially relative to the rotator and the outer housing in the opposite direction to the initial relative movement. As a result, the protrusion will move back along the second pathway and into the third pathway 38. The final position of the protrusion after injection would typically be at or near the position shown in a dashed line and indicated with the reference numeral 63 in FIG. 6. The third pathway is optional but can be beneficial, for example because the third pathway can allow the medicament delivery member guard to extend again after medicament delivery to cover the medicament delivery member, and because it can also provide a medicament delivery member guard lock out after use.

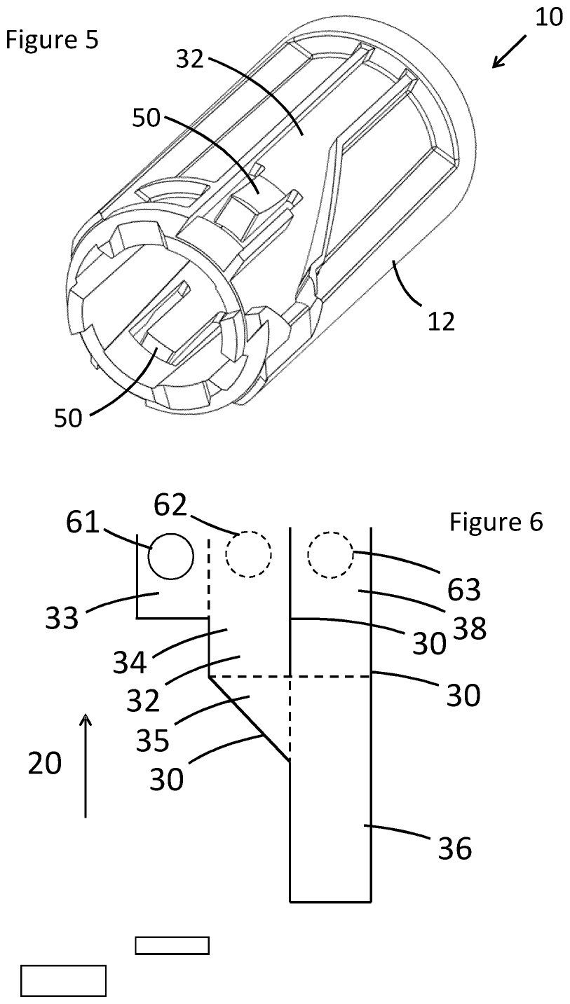

The rotator 10 extends in an axial direction 20 relative to an axis 22 and in a circumferential direction 24 around the axis. In the Figures, the rotator is shown extending entirely around the axis in the circumferential direction, so 360 degrees around the axis, but the rotator could also extend only part of the way round the axis. Various other structural features of the rotator can be seen in the examples, but these features are not essential to the invention described herein. The rotator could be made as a single integral piece or as two or more pieces joined together. The rotator is shown as comprising two tracks (two labyrinths) opposite one another relative to the axis 22 (see FIG. 5 for example), but one, three or more tracks could be provided on the rotator.

The ridge 30 can be one single ridge or alternatively two or more separate portions adjacent to one another or spaced apart from one another. Some or all of the ridge or ridges can have further functions, such as providing structural support for the rotator. The ridge in FIG. 1 could be considered to be two separate portions (one straight section between the two pathways and another larger section extending around the two pathways). The ridge in FIG. 1 is shown as a series of straight sections, but the specific shape of the ridge shown in FIG. 1 is not essential; for example, curved portions could be used instead. The track 32 is shown extending from the distal end of the rotator to the proximal end of the rotator, but the track can also be spaced apart from the distal end of the rotator and/or the proximal end of the rotator. In general, the track described in this application can be considered as a volume of space in which the protrusion can move bounded by the ridge. The surface of the track (which is the surface of the rotator adjacent to the track) would typically also limit movement of the protrusion in practice, although this is not necessarily required for the invention to function. When assembled within a complete medicament delivery device, the track would typically also be physically bounded opposite the surface of the track, e.g. by an outer housing, although this is also not necessarily needed to keep the protrusion within the track. The pathway at the distal end of the track is typically aligned in the axial direction with only one of the two pathways at the proximal end of the track. Any particular ridge portion is typically optional, as limitations to the movement of the rotator and the movement of the medicament delivery member guard can also be provided by other features of a medicament delivery device, such as features of a housing of a medicament delivery device.

The opening 49 in the surface of the track allows the tongue 50 to move relative to the rest of the rotator. The surface of the track is the surface of the rotator between the ridges that form the shape of the track. In the case of the example shown in FIG. 1, the track is on the outside surface of the rotator, i.e. the surface of the rotator facing away from the axis 22, with the protrusion 61 extending from the medicament delivery member guard towards the axis in the radial direction. Alternatively, the track could be on the inside surface of the rotator, with the protrusion extending from the medicament delivery member guard away from the axis in the radial direction.

The tongue 50 deforms in the radial direction. However, in some examples, the tongue could additionally or alternatively be deformed in the circumferential direction and/or the axial direction, depending for example on the shape of the tongue, the location of the tongue in the track and the shape of the protrusion.

A tongue 50 can be provided in an opening 49 in the track, as shown for example in FIGS. 1 and 2. The tongue would typically be partially or entirely in the third pathway. The tongue 50 is designed to only allow the protrusion 61 to move in one direction, and not in the other. Once the protrusion has gone past the tongue 50 (so that the protrusion is in the third pathway 38 in a position closer to the proximal end 14 of the rotator than the position of the tongue), the protrusion is unable to pass back in the distal direction as the tongue blocks it. This can provide a medicament delivery member guard lock, which can shield the medicament delivery member after use and avoid issues such as needle stick injuries.

The tongue typically extends primarily in the axial direction as shown in FIG. 1, for example, extending from a proximal end of the tongue to a distal end of the tongue, though it could also extend in other directions. In the example in FIG. 1, the distal end of the tongue is attached to the rest of the rotator, with the proximal end free to move relative to the rest of the rotator. Various alternatives are available instead of this particular solution. For example, the proximal end could be fixed to the rotator with the distal end free to move, or the tongue could extend in the circumferential direction rather than the distal direction.

The location of the tongue 50 may be varied depending on the shape and relative location of other parts of a medicament delivery device, as different devices may require a somewhat different location to provide a click at the right time during injection (a final click at or near the end of injection). The proximal end and the distal end of the tongue may therefore be in different locations than those shown in the Figures.

The medicament delivery member guard comprises a protrusion 61, which may take various shapes other than the shapes shown in the examples in the Figures. Optionally, the distal face of the protrusion is curved (for example semi-circular as shown in FIG. 8), and the proximal face of the protrusion is pointed, with two linear surfaces either side of the point (as shown in FIG. 8). This can allow the distal face of the protrusion to interact with a curved surface of the ridge portion 70, and one of the linear faces of the proximal face of the protrusion to interact with a linear surface of the ridge portion 73. One alternative is a circular protrusion as shown in FIG. 6.

In general, the medicament delivery member guard is blocked (or at least limited) from moving in the proximal direction relative to the rotator in a completed medicament delivery device until some kind of block is removed; for example, this block could be provided by a feature of the cap (such as the rib 92 and groove 66 described above, or another part of the cap) or could be provided by a part of another medicament delivery device component such as an activation button. Once the block is removed, the medicament delivery member guard is able to move in the proximal direction relative to the rotator.

Alternatively, rather than using cap removal to move the medicament delivery member guard in the proximal direction to move the protrusion 61 from the first section of the first pathway to the second section of the first pathway, this could be effected by interaction with another component or by the user pulling on the medicament delivery member guard after removing the cap. Another alternative is that a medicament delivery member guard spring (not shown), which is arranged between the medicament delivery member guard and the housing for example, pushes the medicament delivery member guard in the proximal direction relative to the housing after the cap is removed (this same spring could be responsible for pushing the medicament delivery member guard in the proximal direction after completion of medicament delivery).

Various modifications to the embodiments described are possible and will occur to those skilled in the art without departing from the invention which is defined by the following claims.

1-15. (canceled)

16. A rotator for a medicament delivery device, the rotator comprising:
a tubular body extending from a proximal end to a distal end in an axial direction relative to an axis and in a circumferential direction relative to the axis; and
one or more ridges extending from a surface of the tubular body, the one or more ridges defining a track on the surface of the tubular body, the track extending in the axial direction from a distal end of the track to a proximal end of the track,
wherein the track comprises a first pathway and a second pathway connected to the first pathway, wherein the first pathway is at the proximal end of the track and the second pathway is at the distal end of the track,
wherein the first pathway comprises two sections adjacent to one another in the circumferential direction, and
wherein a first section of the two sections is delimited in the distal direction by a portion of the one or more ridges, and a second section of the two sections is connected to the second pathway at a distal end of the second section.

17. The rotator of claim 16, wherein the one or more ridges comprise a protrusion extending in the proximal direction, the protrusion extending between the first section of the first pathway and the second section of the first pathway.

18. The rotator of claim 16, wherein the portion of the one or more ridges extends in the circumferential direction.

19. The rotator of claim 16, wherein the portion of the one or more ridges extends perpendicular to the axis.

20. The rotator of claim 16, wherein a proximal face of the portion of the one or more ridges is curved.

21. The rotator of claim 16, wherein the portion of the one or more ridges is a first portion, and the one or more ridges comprises a second portion arranged proximal to the first portion.

22. The rotator of claim 21, wherein the second portion is angled relative to the axis.

23. The rotator of claim 21, wherein the second portion extends from a proximal end to a distal end, and wherein the proximal end of the second portion is adjacent to the second section (34) of the first pathway.

24. The rotator of claim 21, wherein the second portion is linear.

25. The rotator of claim 16, wherein the rotator comprises a third pathway connected to the second pathway, wherein the third pathway is at the proximal end of the track.

26. The rotator of claim 25, wherein the first pathway and the third pathway are separated by at least one of the one or more ridges.

27. The rotator of claim 16, wherein the one or more ridges extend from an outer surface of the rotator.

28. A medicament delivery device comprising the rotator of claim 16.

29. The medicament delivery device of claim 28, wherein the medicament delivery device comprises a medicament delivery member guard, the medicament delivery member guard comprising a protrusion, wherein the protrusion is in the track of the rotator.

30. The medicament delivery device of claim 29, wherein the protrusion is configured to be in the first section of the first pathway prior to device activation, in the second section of the first pathway after device activation and in the second pathway during medicament delivery.
